SEQUENCE LISTING (50 sequences)

SEQ ID NO: 1 - DNA, 846 bp, Homo sapiens
gggggcgtcggggcccggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 2 - protein, 282 aa, Homo sapiens
GGVGARREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 3 - DNA, 663 bp, Homo sapiens
ccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 4 - protein, 221 aa, Homo sapiens
PDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 5 - DNA, 642 bp, Homo sapiens
aagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 6 - protein, 214 aa, Homo sapiens
KRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 7 - DNA, 636 bp, Homo sapiens
tgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 8 - protein, 212 aa, Homo sapiens
CGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 9 - DNA, 510 bp, Homo sapiens
ttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 10 - protein, 170 aa, Homo sapiens
FSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 11 - DNA, 417 bp, Homo sapiens
gtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 12 - protein, 139 aa, Homo sapiens
VDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 13 - DNA, 405 bp, Homo sapiens
acgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 14 - protein, 135 aa, Homo sapiens
TLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 15 - DNA, 864 bp, Homo sapiens
gggggcgtcggggcccggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 16 - protein, 288 aa, Homo sapiens
GGVGARREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 17 - DNA, 681 bp, Homo sapiens
ccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 18 - protein, 227 aa, Homo sapiens
PDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 19 - DNA, 660 bp, Homo sapiens
aagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 20 - protein, 220 aa, Homo sapiens
KRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 21 - DNA, 654 bp, Homo sapiens
tgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 22 - protein, 218 aa, Homo sapiens
CGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 23 - DNA, 528 bp, Homo sapiens
ttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 24 - protein, 176 aa, Homo sapiens
FSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 25 - DNA, 435 bp, Homo sapiens
gtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 26 - protein, 145 aa, Homo sapiens
VDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 27 - DNA, 423 bp, Homo sapiens
acgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 28 - protein, 141 aa, Homo sapiens
TLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 29 - DNA, 906 bp, Homo sapiens
atgcggcgctgggcctgggccgcggtcgtggtcctcctcgggccgcagctcgtgctcctcgggggcgtcggggcccggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 30 - protein, 302 aa, Homo sapiens
MRRWAWAAVVVLLGPQLVLLGGVGARREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 31 - DNA, 924 bp, Homo sapiens
atgcggcgctgggcctgggccgcggtcgtggtcctcctcgggccgcagctcgtgctcctcgggggcgtcggggcccggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 32 - protein, 308 aa, Homo sapiens
MRRWAWAAVVVLLGPQLVLLGGVGARREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 33 - DNA, 831 bp, Homo sapiens
cggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacg

SEQ ID NO: 34 - protein, 277 aa, Homo sapiens
RREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGT

SEQ ID NO: 35 - DNA, 849 bp, Homo sapiens
cggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgcaccatcaccatcaccat

SEQ ID NO: 36 - protein, 283 aa, Homo sapiens
RREAQRTQQPGQRADPPNATASASSREGLPEAPKPSQASGPEFSDAHMTWLNFVRRPDDGALRKRCGSRDKKPRDLFGPPGPPGAEVTAETLLHEFQELLKEATERRFSGLLDPLLPQGAGLRLVGEAFHCRLQGPRRVDKRTLVELHGFQAPAAQGAFLRGSGLSLASGRFTAPVSGIFQFSASLHVDHSELQGKARLRARDVVCVLICIESLCQRHTCLEAVSGLESNSRVFTLQVQGLLQLQAGQYASVFVDNGSGAVLTIQAGSSFSGLLLGTHHHHHH

SEQ ID NO: 37 - DNA, 24 bp, Artificial Sequence
  Primer INSP163-CP1
tgagccgcctcgggacggagccat

SEQ ID NO: 38 - DNA, 24 bp, Artificial Sequence
  Primer INSP163-CP2
acgtgcccaggagcagcccggaga

SEQ ID NO: 39 - DNA, 35 bp, Artificial Sequence
  Primer INSP163-EX1
gcaggcttcgccaccatgcggcgctgggcctgggc

SEQ ID NO: 40 - DNA, 35 bp, Artificial Sequence
  Primer INSP163-EX2
tgatggtgatggtgcgtgcccaggagcagcccgga

SEQ ID NO: 41 - DNA, 37 bp, Artificial Sequence
  Primer GCP Forward
ggggacaagtttgtacaaaaaagcaggcttcgccacc

SEQ ID NO: 42 - DNA, 51 bp, Artificial Sequence
  Primer GCP Reverse
ggggaccactttgtacaagaaagctgggtttcaatggtgatggtgatggtg

SEQ ID NO: 43 - DNA, 20 bp, Artificial Sequence
  Primer pEAK12F
gccagcttggcacttgatgt

SEQ ID NO: 44 - DNA, 20 bp, Artificial Sequence
  Primer pEAK12R
gatggaggtggacgtgtcag

SEQ ID NO: 45 - DNA, 18 bp, Artificial Sequence
  Primer 21M13
tgtaaaacgacggccagt

SEQ ID NO: 46 - DNA, 18 bp, Artificial Sequence
  Primer M13REV
caggaaacagctatgacc

SEQ ID NO: 47 - DNA, 19 bp, Artificial Sequence
  Primer T7
taatacgactcactatagg

SEQ ID NO: 48 - DNA, 18 bp, Artificial Sequence
  Primer T3
attaaccctcactaaagg

SEQ ID NO: 49 - DNA, 20 bp, Artificial Sequence
  Primer h-INSP163-exon3-4-331F
gaagtgaccgcggagactct

SEQ ID NO: 50 - DNA, 17 bp, Artificial Sequence
  h-INSP163-exon3-4-392R
cgctccgtggcctcttt

(19) United States
(12) Patent Application Publication
(10) Pub. No.: US 2009/0035274 A1
(43) Pub. Date: Feb. 5, 2009

(54) TREATMENT AND/OR PREVENTION OF CANCER AND/OR ARTHRITIS

(75) Inventors: Christine Power, Thoiry (FR); Melanie Yorke-Smith, Confignon (GB)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Laboratoires Serono SA, Coinsins , Vaud (CH)

(21) Appl. No.: 11/912,432

(22) PCT Filed: Apr. 20, 2006
(86) PCT No.: PCT/EP2006/061710

§ 371 Date: Oct. 24, 2007

Related U.S. Application Data
(60) Provisional application No. 60/681,731, filed on May 17, 2005.

(30) Foreign Application Priority Data
Apr. 26, 2005 (EP) 05103402.3

(51) Int. Cl. A61K 38/21; A61K 38/00; A61K 31/7088; A61P 31/00; A61P 19/00; A61K 39/395; A61K 35/12

(52) U.S. Cl. 424/85.6; 514/12; 514/44; 514/8; 424/133.1; 424/93.21; 424/85.4

(57) ABSTRACT

The invention relates to the use of INSP163 for treatment and/or prevention of cancer and/or musculoskeletal/connective tissue disorder, in particular of lung cancer and/or osteoarthritis. Combinations of INSP163 with an interferon, a TNF antagonist or a further anti-cancer or anti-arthritis agent are also within the present invention.

### FIELD OF THE INVENTION

The invention relates to the use of INSP163 for the treatment and/or prevention of cancer and/or musculoskeletal/connective tissue disorder, in particular lung cancer and/or osteoarthritis.

### BACKGROUND OF THE INVENTION

Osteoarthritis (Degenerative Joint Disease; Osteoarthrosis; Hypertrophic Osteoarthritis) is an arthropathy with altered hyaline cartilage and characterized by loss of articular cartilage and hypertrophy of bone, producing osteophytes. Osteoarthritis (OA), the most common articular disorder, begins asymptomatically in the 2nd to 3rd decades and is extremely common by age 70. Almost all persons by age 40 have some pathologic change in weight-bearing joints, although relatively few have symptoms. Men and women are equally affected, but onset is earlier in men.

There is no evidence that widely used NSAIDs have any long-term benefit on OA. Acetaminophen in doses of up to 1 g qid is a useful analgesic and is generally safer than NSAIDs. In patients with refractory pain or with more signs of inflammation, aspirin or other NSAIDs may be used and may provide better relief of symptoms under Rheumatoid Arthritis. COX-2 inhibitors (also called coxibs) control inflammation and decrease pain with fewer GI side effects; however, one member of the class, rofecoxib (withdrawn from market), appears to increase the risk of cardiovascular events after long-term use. The risk for cardiovascular events with other coxibs is undergoing evaluation. Because one study has shown a 2.5-fold increase in cardiovascular events with celecoxib, FDA recommendations, pending further evidence, are to limit use of any coxib to patients who are at a high risk of GI bleeding, have a history of intolerance to nonselective NSAIDs, or are not doing well on nonselective NSAIDs. Use of coxibs for long periods or in patients with cardiovascular risk factors should be approached cautiously.

Muscle relaxants (usually in low doses) occasionally provide temporary benefit when pain arises from muscles strained by attempting to support OA joints. Oral corticosteroid therapy usually is not indicated. Intra-articular depot corticosteroids are helpful when effusions or signs of inflammation are present; these drugs usually are needed only intermittently and should generally be used as infrequently as possible. Drug therapy is the least important aspect of optimum management, perhaps 15% of a total program. Hyaluronic acid, a normal physiologic component of synovial fluid, has proven effective in the management of OA of the knee. Commercial preparations, Hyalgan and ARTZ, by injection, resulted in measurable improvement using clinical, radiologic, and laboratory criteria.

Bronchogenic Carcinoma is a highly malignant primary lung tumor that accounts for most cases of lung cancer and has a very poor prognosis. Bronchogenic carcinoma accounts for >90% of all lung tumors. It is the second most common cancer in men (13%) and the third most common cancer in women (13%). It is the leading cause of cancer death among men (32%) and women (25%), and its incidence appears to be rising more rapidly among women. It is most common between the ages of 45 and 70.

Four histologic types of bronchogenic carcinoma usually are distinguished: squamous cell, commonly arising in the larger bronchi and spreading by direct extension and lymph node metastasis; undifferentiated small cell, often associated with early hematogenous metastases; undifferentiated large cell, usually spreading through the bloodstream; and adenocarcinoma, commonly peripheral, often spreading through the bloodstream. All types also commonly spread via the lymphatics.

Bronchioloalveolar carcinoma, a subtype of adenocarcinoma, consolidates airspaces and often does not extend beyond the lungs. Although a solitary form exists, this cancer is sometimes distinguished from other types of bronchogenic carcinoma by its multifocal origin.

Horner's syndrome (due to invasion of the cervical thoracic sympathetic nerves) consists of enopthalmos, miosis, ptosis, and ipsilateral facial anhidrosis.

Pancoast syndrome (due to infiltration of the brachial plexus and neighboring ribs and vertebrae) consists of pain, numbness, and weakness of the affected arm. The two syndromes may coexist.

Paraneoplastic syndromes of lung cancer, which are numerous, are extrapulmonary, remote effects of tumors. They lead to metabolic and neuromuscular disturbances unrelated to the primary tumor or metastases. They may be the first sign of occurrence or recurrence, but they do not necessarily indicate that a tumor has spread outside the chest. In hypertrophic pulmonary osteoarthropathy (the best known), clubbing of the fingers and toes and periosteal elevation of the distal parts of long bones occur. All levels of the nervous system may be affected—principally causing encephalopathy, subacute cerebellar degeneration, encephalomyelitis, the Eaton-Lambert syndrome, and peripheral neuropathy. Polymyositis and dermatomyositis or metabolic syndromes due to production of substances with hormonal activity may develop. Small cell carcinomas may secrete ectopic ACTH, resulting in Cushing's syndrome, or ADH, causing water retention and hyponatremia, and are also associated with the carcinoid syndrome (flushing, wheezing, diarrhea, and cardiac valvular lesions). Squamous cell carcinomas may secrete parathyroid hormone-like substances that produce hypercalcemia. Other endocrine syndromes associated with primary lung carcinomas include gynecomastia, hyperglycemia, thyrotoxicosis, and skin pigmentation. Hematologic disorders, including thrombocytopenic purpura, leukemoid reaction, myelophthisic anemia, polycythemia, and marantic thrombosis, may also occur.

Squamous cell carcinoma is a common form of lung cancer, accounting for approximately one-third of all cases of bronchogenic carcinomas. Unlike adenocarcinoma, it is strongly linked with a history of cigarette smoking. Its histogenesis may be related to chronic inflammation and injury of the bronchial epithelium, which leads to replacement of the normal ciliated columnar epithelium by a squamous epithelium. This transformation from a glandular epithelium to squamous epithelium is known as squamous metaplasia.

Bronchogenic carcinoma has a poor prognosis. On average, patients with untreated bronchogenic carcinoma survive 8 months; about 10 to 35% of tumors are resectable, but the overall 5-year survival rate is approximately 13%. In patients with well-circumscribed, slow-growing tumors, the 5-year survival rate after excision ranges from 15% in patients with stage IIIA non-small cell carcinoma to 70% in patients with stage I non-small cell carcinoma. Best results are obtained in patients with peripheral nodular lesions treated by lobectomy. Second primary lung cancers develop in 6 to 12% of survivors. Because small cell carcinoma has almost always spread beyond the primary site at the time of diagnosis, it is usually inoperable. Rarely, early-stage small cell carcinoma can be surgically resected, but because tumors are likely to recur, adjuvant chemotherapy with cisplatin and etoposide is usually recommended. A second primary cancer develops after treatment of early-stage small cell carcinoma in 25 to 50% of cases.

Prevention of bronchogenic carcinoma includes avoiding tobacco and exposure to potentially carcinogenic substances in industry.

Chemotherapy with multiple drugs, particularly cisplatin and topoisomerase inhibitors—with or without radiation therapy—has yielded higher survival rates than surgery has in patients with small cell carcinoma; cures are rare. Chemotherapy in unresectable stage IIIA, IIIB, or IV non-small cell lung carcinomas appears to improve median survival by 6 to 12 weeks on average and can effectively ameliorate symptoms of the disease in patients who respond. Drugs active in this disease include platinum compounds (cisplatin and carboplatin), vinca alkaloids (vinorelbine, vincristine, and vinblastine), taxines (docetaxel and paclitaxel), and various topoisomerase inhibitors.

The protein INSP163 was disclosed in International Application No. PCT/GB2004/004544 as a secreted protein containing a jelly-roll fold, in particular, as a member of the TNF (tumor necrosis factor)-like family of cytokines, specifically as a c1q-like protein. No experimental data have been provided so far to show any involvement of INSP163 in cancer and/or musculoskeletal/connective tissue disorder.

### SUMMARY OF THE INVENTION

The invention is based on the unexpected finding that INSP163 displays restricted expression in specific tissues, namely in lung tumor and osteoarthritis tissues.

It is therefore a first object of the invention to use INSP163 for the preparation of a medicament for the treatment and/or prevention of cancer and/or musculoskeletal/connective tissue disorder, in particular of lung cancer and/or osteoarthritis. It is a second object of the invention to use a cell expressing INSP163, or an expression vector comprising the coding sequence of INSP163, for the preparation of a medicament for the treatment and/or prevention of cancer and/or musculoskeletal/connective tissue disorder, in particular of lung cancer and/or osteoarthritis. The present invention is also directed towards a pharmaceutical composition comprising INSP163 for the treatment and/or prevention of cancer and/or musculoskeletal/connective tissue disorder, in particular of lung cancer and/or osteoarthritis.

### DESCRIPTION OF THE INVENTION

The invention is based on the unexpected finding of the restricted expression of INSP163 in one lung tumor tissue and in two osteoarthritis tissues. The lung tumor is a bronchogenic carcinoma, more specifically a squamous cell carcinoma. This specific pattern of expression of INSP163 leads to the conclusion of the involvement of INSP163 in lung cancer and/or osteoarthritis. These surprising properties presently characterized of the polynucleotides or the corresponding polypeptides of International Application No. PCT/GB2004/004544 make them particularly suitable for the preparation of a medicament or pharmaceutical composition.

In a first aspect, the invention therefore relates to the use of a polypeptide for the preparation of a medicament for the treatment and/or prevention of cancer and/or musculoskeletal/connective tissue disorder, wherein said polypeptide is selected from the group consisting of:
a) A polypeptide consisting of SEQ ID NO: 30, or
b) A polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36, or
c) A mature form of the polypeptide consisting of SEQ ID NO: 2 or SEQ ID NO: 34, or
d) A histidine tag form of the polypeptide consisting of SEQ ID NO: 16, SEQ ID NO: 18, SEQ ID NO: 20, SEQ ID NO: 22, SEQ ID NO: 24, SEQ ID NO: 32, SEQ ID NO: 26, SEQ ID NO: 28, SEQ ID NO: 32, or SEQ ID NO: 36, or
e) A cleaved form of the polypeptide consisting of SEQ ID NO: 4, SEQ ID NO: 6, SEQ ID NO: 8, SEQ ID NO: 10, SEQ ID NO: 12, or SEQ ID NO: 14, or
f) A glycosylated form of the polypeptide, wherein the polypeptide is glycosylated at one or more sites, or
g) A mutein of any of (a) to (f), wherein the amino acid sequence has at least 40% or 50% or 60% or 70% or 80% or 90% identity to at least one of the sequences in (a) to (f), retaining INSP163 biological activity, or
h) A mutein of any of (a) to (f) wherein any changes in the amino acid sequence are conservative amino acid substitutions to the amino acid sequences in (a) to (f), retaining INSP163 biological activity, or
i) A salt or an isoform, fusion protein, functional derivative, active fraction or circularly permutated derivative of any of (a) to (h).

In a second aspect, the invention relates to the use of a nucleic acid molecule for the preparation of a medicament for the treatment and/or prevention of a cancer and/or musculoskeletal/connective tissue disorder, wherein said nucleic acid is selected from the group consisting of:
a) A nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35, or
b) A nucleic acid sequence which hybridizes to the complement of the nucleic acid sequence of (a) under moderately stringent conditions or under highly stringent conditions, or
c) A nucleic acid sequence of any of (a) or (b) wherein said nucleic acid sequence encodes an amino acid sequence having conservative amino acid substitutions to the amino acid sequences in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

In a third aspect, the invention relates to a pharmaceutical composition for the treatment and/or prevention of cancer and/or musculoskeletal/connective tissue disorder comprising a polypeptide selected from the group consisting of:
a) A polypeptide consisting of SEQ ID NO: 30, or
b) A polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36, or
c) A mature form of the polypeptide consisting of SEQ ID NO: 2 or SEQ ID NO: 34, or
d) A histidine tag form of the polypeptide consisting of SEQ ID NO: 16, SEQ ID NO: 18, SEQ ID NO: 20, SEQ ID NO: 22, SEQ ID NO: 24, SEQ ID NO: 32, SEQ ID NO: 26, SEQ ID NO: 28, SEQ ID NO: 32, or SEQ ID NO: 36, or
e) A cleaved form of the polypeptide consisting of SEQ ID NO: 4, SEQ ID NO: 6, SEQ ID NO: 8, SEQ ID NO: 10, SEQ ID NO: 12, or SEQ ID NO: 14, or
f) A glycosylated form of the polypeptide, wherein the polypeptide is glycosylated at one or more sites, or
g) A mutein of any of (a) to (f), wherein the amino acid sequence has at least 40% or 50% or 60% or 70% or 80% or 90% identity to at least one of the sequences in (a) to (f), retaining INSP163 biological activity, or
h) A mutein of any of (a) to (f) wherein any changes in the amino acid sequence are conservative amino acid substitutions to the amino acid sequences in (a) to (f), retaining INSP163 biological activity, or
i) A salt or an isoform, fusion protein, functional derivative, active fraction or circularly permutated derivative of any of (a) to (h).

In a fourth aspect, the invention relates to a pharmaceutical composition for the treatment and/or prevention of a cancer and/or musculoskeletal/connective tissue disorder comprising a nucleic acid selected from the group consisting of:
a) A nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35, or
b) A nucleic acid sequence which hybridizes to the complement of the nucleic acid sequence of (a) under moderately stringent conditions or under highly stringent conditions, or
c) A nucleic acid sequence of any of (a) or (b) wherein said nucleic acid sequence encodes an amino acid sequence having conservative amino acid substitutions to the amino acid sequences in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

It will be appreciated by the person skilled in the art that in accordance with the present invention, a substance which stimulates release or potentiates the activity of endogenous INSP163 can equally be used for treatment and/or prevention of cancer and/or musculoskeletal/connective tissue disorder, in particular of lung cancer and/or osteoarthritis.

The polypeptide having the sequence recited in SEQ ID NO: 2 is referred to hereafter as the “alternative mature INSP163 polypeptide”. The polypeptide having the sequence recited in SEQ ID NO: 34 is referred to hereafter as the “mature INSP163 polypeptide”. SEQ ID NO: 2 or SEQ ID NO: 34 refer herein to a “mature form”. The polypeptide having the sequence recited in SEQ ID NO: 4 is referred to hereafter as the “INSP163-A polypeptide”. The polypeptide having the sequence recited in SEQ ID NO: 6 is referred to hereafter as the “INSP163-B polypeptide”. The polypeptide having the sequence recited in SEQ ID NO: 8 is referred to hereafter as the “INSP163-C polypeptide”. The polypeptide having the sequence recited in SEQ ID NO: 10 is referred to hereafter as the “INSP163-D polypeptide”. The polypeptide having the sequence recited in SEQ ID NO: 12 is referred to hereafter as the “INSP163-E polypeptide”. The polypeptide having the sequence recited in SEQ ID NO: 14 is referred to hereafter as the “INSP163-F polypeptide”. SEQ ID NO: 4, SEQ ID NO: 6, SEQ ID NO: 8, SEQ ID NO: 10, SEQ ID NO: 12, or SEQ ID NO: 14 refer herein to a “cleaved form”.

Although the Applicant does not wish to be bound by this theory, it is postulated that the INSP163 polypeptide further comprises a signal peptide at the N-terminus that is 25 amino acids in length leading to a mature INSP163 polypeptide.

Furthermore, it is postulated that the INSP163 polypeptide further comprises a signal peptide at the N-terminus that is 20 amino acids in length leading to an alternative mature INSP163 polypeptide.

The mature INSP163 polypeptide and alternative mature INSP163 polypeptide sequence with their respective postulated signal sequence is recited in SEQ ID NO: 30.

The polypeptide having the sequence recited in SEQ ID NO: 30 is referred to hereafter as “the INSP163 polypeptide”.

The polypeptides of the invention may further comprise a histidine tag and are herein referred to as a “histidine tag form”. Preferably the histidine tag is found at the C-terminus of the polypeptide. Preferably the histidine tag comprises 1-10 histidine residues (e.g. 1, 2, 3, 4, 5, 6, 7, 8, 9 or 10 residues). More preferably, the histidine tag comprises 6 residues. Preferred polypeptides are therefore those comprising the sequence recited in SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 32 and/or SEQ ID NO: 36.

The polypeptide having the sequence recited in SEQ ID NO: 16 is referred to hereafter as the “histidine tag alternative mature INSP163 polypeptide”. The polypeptide having the sequence recited in SEQ ID NO: 18 is referred to hereafter as the “histidine tag INSP163-A polypeptide”. The polypeptide having the sequence recited in SEQ ID NO: 20 is referred to hereafter as the “histidine tag INSP163-B polypeptide”. The polypeptide having the sequence recited in SEQ ID NO: 22 is referred to hereafter as the “histidine tag INSP163-C polypeptide”. The polypeptide having the sequence recited in SEQ ID NO: 24 is referred to hereafter as the “histidine tag INSP163-D polypeptide”. The polypeptide having the sequence recited in SEQ ID NO: 26 is referred to hereafter as the “histidine tag INSP163-E polypeptide”. The polypeptide having the sequence recited in SEQ ID NO: 28 is referred to hereafter as the “histidine tag INSP163-F polypeptide”. The polypeptide having the sequence recited in SEQ ID NO: 32 is referred to hereafter as the “histidine tag INSP163 polypeptide”. The polypeptide having the sequence recited in SEQ ID NO: 36 is referred to hereafter as the “histidine tag mature INSP163 polypeptide”.

The term “INSP163 polypeptides” or “INSP163” as used herein includes the alternative mature INSP163 polypeptide, the mature INSP163 polypeptide, the INSP163-A polypeptide, the INSP163-B polypeptide, the INSP163-C polypeptide, the INSP163-D polypeptide, the INSP163-E polypeptide, the INSP163-F polypeptide, the INSP163 polypeptide, the histidine tag alternative mature INSP163 polypeptide, the histidine tag INSP163-A polypeptide, the histidine tag INSP163-B polypeptide, the histidine tag INSP163-C polypeptide, the histidine tag INSP163-D polypeptide, the histidine tag INSP163-E polypeptide, the histidine tag INSP163-F polypeptide, the histidine tag INSP163 polypeptide, the histidine tag mature INSP163 polypeptide and the full length INSP163 (mature INSP163 polypeptide with signal peptide).

Preferably, the nucleic acid molecule comprises of the nucleic acid sequence as recited in SEQ ID NO: 1 (encoding the alternative mature INSP163 polypeptide), SEQ ID NO: 3 (encoding the INSP163-A polypeptide), SEQ ID NO: 5 (encoding the INSP163-B polypeptide), SEQ ID NO: 7 (encoding the INSP163-C polypeptide), SEQ ID NO: 9 (encoding the INSP163-D polypeptide), SEQ ID NO: 11 (encoding the INSP163-E polypeptide), SEQ ID NO: 13 (encoding the INSP163-F polypeptide), SEQ ID NO: 15 (encoding the histidine tag alternative mature INSP163 polypeptide), SEQ ID NO: 17 (encoding the histidine tag INSP163-A polypeptide), SEQ ID NO: 19 (encoding the histidine tag INSP163-B polypeptide), SEQ ID NO: 21 (encoding the histidine tag INSP163-C polypeptide), SEQ ID NO: 23 (encoding the histidine tag INSP163-D polypeptide), SEQ ID NO: 25 (encoding the histidine tag INSP163-E polypeptide), SEQ ID NO: 27 (encoding the histidine tag INSP163-F polypeptide), SEQ ID NO: 29 (encoding the INSP163 polypeptide), SEQ ID NO: 31 (encoding the histidine tag INSP163 polypeptide), SEQ ID NO: 33 (encoding the mature INSP163 polypeptide), SEQ ID NO: 35 (encoding the histidine tag mature INSP163 polypeptide).

Preferably, the nucleic acid molecule consists of the nucleic acid sequence recited in SEQ ID NO: 1 (encoding the alternative mature INSP163 polypeptide), SEQ ID NO: 3 (encoding the INSP163-A polypeptide), SEQ ID NO: 5 (encoding the INSP163-B polypeptide), SEQ ID NO: 7 (encoding the INSP163-C polypeptide), SEQ ID NO: 9 (encoding the INSP163-D polypeptide), SEQ ID NO: 11 (encoding the INSP163-E polypeptide), SEQ ID NO: 13 (encoding the INSP163-F polypeptide), SEQ ID NO: 15 (encoding the histidine tag alternative mature INSP163 polypeptide), SEQ ID NO: 17 (encoding the histidine tag INSP163-A polypeptide), SEQ ID NO: 19 (encoding the histidine tag INSP163-B polypeptide), SEQ ID NO: 21 (encoding the histidine tag INSP163-C polypeptide), SEQ ID NO: 23 (encoding the histidine tag INSP163-D polypeptide), SEQ ID NO: 25 (encoding the histidine tag INSP163-E polypeptide), SEQ ID NO: 27 (encoding the histidine tag INSP163-F polypeptide), SEQ ID NO: 29 (encoding the INSP163 polypeptide), SEQ ID NO: 31 (encoding the histidine tag INSP163 polypeptide), SEQ ID NO: 33 (encoding the mature INSP163 polypeptide), SEQ ID NO: 35 (encoding the histidine tag mature INSP163 polypeptide).

The term “treatment and/or prevention” as used herein encompasses any attenuation, reduction, or partial, substantial or complete prevention or blockage of disease formation, development, progression or of the formation, development or progression of any one or several or all of the symptoms of the disease.

Preferably, “cancer” is selected among cancer from blood and lymphatic systems, skin cancers, cancer of digestive systems, cancers of urinary systems, breast cancer, ovarian cancer, gynaecological cancers, choriocarcionoma, lung cancer, Brain Tumors, Bone Tumors, Carcinoid Tumor, Nasopharyngeal Cancer, Retroperitoneal sarcomas, Soft Tissue Tumors, Thyroid Cancer or Cancers of Unknown Primary Site.

Preferably, “tumors of the lungs” or “lung cancer”, as used interchangeably herein, are selected from benign or malignant primary tumors or from metastases from primary cancers of many other organs and tissues.

Preferably the lung cancer is selected from primary lung tumors including bronchogenic carcinoma, bronchial carcinoid, chondromatous hamartoma (benign), solitary lymphoma, sarcoma (malignant) or multifocal lymphomas.

Preferably, the bronchogenic carcinoma is selected from squamous cell carcinoma, undifferentiated small cell carcinoma, undifferentiated large cell carcinoma, adenocarcinoma or Bronchioloalveolar carcinoma.

Preferably, the bronchogenic carcinoma is squamous cell carcinoma or non-small cell lung carcinoma.

Preferably the lung cancer is selected from metastases from primary cancers of the skin, breast, colon, prostate, kidney, thyroid, stomach, cervix, rectum, testis, and bone and from melanoma.

“Bronchial carcinoid” or “bronchial adenoma”, terms that can be used interchangeably, may be benign or malignant and occurs equally in both sexes. Its course is prolonged. The endobronchial portion of the tumor may obstruct the lumen of major bronchi. Brisk bleeding from the overlying mucous membrane often occurs. Recurrent pneumonia within the same lung zone and localized overlying pleural pain are common. Metastases are uncommon but may occur to regional lymph nodes.

Preferably, “Musculoskeletal/Connective Tissue disorders” is a disorder selected from Tumors of Bones and Joints, Diffuse Connective Tissue Disease, Osteoporosis, Arthritis Associated with Spondylitis, Paget's Disease of Bone, Osteoarthritis (OA) and Neurogenic Arthropathy, Nonarticular Rheumatism, Avascular Necrosis, Common Foot and Ankle Disorders, Infections of Bones and Joints, Common Hand Disorders, Crystal-Induced Conditions or Common Sports Injuries.

“Osteoarthritis” (OA) is classified as primary (idiopathic) or secondary to some known cause. Primary generalized OA involves the distal and proximal interphalangeal joints (producing Heberden's and Bouchard's nodes), 1st carpometacarpal joint, intervertebral disks and zygapophyseal joints in the cervical and lumbar vertebrae, 1st metatarsophalangeal joint, hip, and knee. Preferably, “Osteoarthritis” is selected from subsets of primary OA including erosive, inflammatory OA and rapidly destructive OA of shoulders and less often of hips and knees in the elderly. Preferably, “Osteoarthritis” is selected from diffuse idiopathic skeletal hyperostosis, which is a syndrome involving large OA-like spinal osteophytes but little or no loss of articular cartilage, Chondromalacia patellae, a mild OA of patellar cartilage in young people or hypertrophic pulmonary osteoarthropathy.

Preferably, “Osteoarthritis” is selected from a secondary OA derived from congenital joint abnormalities; genetic defects; infectious, metabolic, endocrine, and neuropathic diseases; diseases that alter the normal structure and function of hyaline cartilage (for example rheumatoid arthritis or RA, gout and chondrocalcinosis) and trauma (including fracture) to the hyaline cartilage or surrounding tissue (eg, from prolonged overuse of a joint or group of joints associated with occupations such as foundry work, coal mining, and bus driving).

The term “INSP163” as used herein, relates to a protein comprising all, or a portion of the sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36 (all human) of the enclosed sequence listing, as well as to salts, isoforms, muteins, active fractions, functional derivatives and circularly permutated derivatives thereof. INSP163 from species other than human, such as mouse or rat, may be used in accordance with the present invention, as long as there is a sufficient identity between the proteins as to allow the protein to exhibit its biological activity, and without eliciting a substantial immune response in a human being.

The term “INSP163”, as used herein, further relates to any fragment, portion, domain or sub-domain of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36 showing the desired activity in lung cancer and/or osteoarthritis or other cancer and/or musculoskeletal/connective tissue disorder. Protein fragments, isoforms, differentially glycosylated or sialylated forms or one or more domains of the protein may be used according to the invention, as long as they exhibit any beneficial effect on cancer and/or musculoskeletal/connective tissue disorder, preferably an effect which is at least comparable of the full length protein. The beneficial effect can be measured in one of the in vitro or in vivo tests described in the examples below, or in any other assay adequate to demonstrate an effect in cancer and/or musculoskeletal/connective tissue disorder, in particular of lung cancer and/or osteoarthritis.

In accordance with the present invention, INSP163 can be a naturally occurring, i.e. native protein, or a recombinant protein. Recombinant production may be carried out in eukaryotic cells, such as yeast cells or mammalian cells, preferably in CHO cells, HEK cells (human embryonic kidney cells) or in human fibroblast cells or cell lines. It may further be produced in prokaryotic cells such as E. coli.

Preferably, INSP163 is glycosylated at one or more sites. It may also be unglycosylated, depending on the given needs and the source of production or isolation of the protein.

Preferably, the polypeptides of the invention are glycosylated at residues 43 and/or 281 of SEQ ID NO: 30.

The term “salts” herein refers to both salts of carboxyl groups and to acid addition salts of amino groups of INSP163 molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of INSP163 relevant to the present invention, i.e., exert a beneficial effect on cancer and/or musculoskeletal/connective tissue disorder, in particular lung cancer and/or osteoarthritis.

Isoforms or splice variants of INSP163 may also be used according to the invention, as long as they are capable of inhibiting disease progression and/or symptoms of that disease.

As used herein the term “muteins” refers to analogs of INSP163, in which one or more of the amino acid residues of natural INSP163 are replaced by different amino acid residues, or are deleted, or one or more amino acid residues are added to the natural sequence of INSP163, having preferably at least the same activity as wild type INSP163 or even having a much more potent activity. The biological activity of INSP163 can e.g. be measured by assaying INSP163 in its capacity to induce apoptosis. The assay as described in Example 3 for determining IKK2 activity in non-small cell lung carcinoma cells is suitable to determine if INSP163 can induce cell death. Assays for assessing protein-protein interactions are well known by the person skilled in the art. Examples for such assays are ELISA type binding assays, immuno-precipitation assays, or measurement in any other suitable system such as the BIAcore system. These muteins are prepared by known synthesis and/or by site-directed mutagenesis techniques, or any other known technique suitable therefor.

Any such mutein preferably has a sequence of amino acids sufficiently duplicative of that INSP163, such as to have at least a substantially similar activity of INSP163. The activity of an INSP163 mutant can further be tested in the assays explained in the example below (example 3). Measuring cell death of non-small cell lung carcinoma cells treated with INSP163 may be a suitable test for assessing the activity of INSP163 muteins, for example.

Muteins in accordance with the present invention include proteins encoded by a nucleic acid, such as DNA or RNA, which hybridizes to DNA or RNA, which encodes INSP163, in accordance with the present invention, under stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

Any such mutein preferably has a sequence of amino acids sufficiently duplicative of that of INSP163, such as to have substantially similar, or even better, biological activity as INSP163.

One easily measurable activity of INSP163 is its capability of inducing cell death of cancer cells (e.g. A549 cells). As long as the mutein has substantial activity in iducing cell death of cancer cells (e.g. A549 cells, see Example 3), it can be considered to have substantially similar activity to INSP163. Thus, it can be determined whether any given mutein has at least substantially the same activity as INSP163 by means of routine experimentation comprising subjecting such a mutein.

In a preferred embodiment, any such mutein has at least 40% identity or homology with the sequence of INSP163. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al, 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nim.nih.gov) and FASTA (Pearson W R, 1990; Pearson 1988).

Muteins of INSP163, which can be used in accordance with the present invention, or nucleic acids encoding them, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of INSP163 polypeptides or proteins, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of INSP163 polypeptides or proteins, for use in the present invention include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al).

The term “fusion protein” refers to a polypeptide comprising INSP163, or a mutein thereof, fused with another protein, which, e.g., has an extended residence time in body fluids. Fusion proteins comprising all or a functional part of INSP163 fused to all or a functional part of a protein capable of improving the biological activities of the molecule, like half-life in the human body, for instance, are preferred according to the invention. In a preferred embodiment the fusion protein comprises an immunoglobulin (Ig) fusion. Fusion proteins comprising all or part of INSP163 fused to all or part of an immunoglobulin are highly preferred. They can be monomeric or multimeric, hetero- or homomultimeric. Advantageously, the fusion protein comprises the constant region of an immunoglobulin, in particular of the Fc portion of the immunoglobulin. Embodiments in which the immunoglobulin is of the IgG1 or IgG2 isotype are further preferred according to the invention. Preferably, the fusion is an Fc fusion.

INSP163 may thus be fused to another protein, polypeptide or the like, e.g., an immunoglobulin or a fragment thereof. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the INSP163 sequence and the immunoglobulin sequence.

“Functional derivatives” as used herein cover derivatives of INSP163, and their muteins and fusion proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is at least substantially similar to the activity of INSP163, and do not confer toxic properties on compositions containing it. Therefore, in a preferred embodiment the functional derivative comprises at least one moiety attached to one or more functional groups, which occur as one or more side chains on the amino acid residues.

In accordance with the present invention, polyethylene glycol (PEG) side-chains are highly preferred moieties. PEG side chains may mask antigenic sites and extend the residence of the substance they are attached to in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

“Active fractions” of INSP163 and its muteins and fusion proteins, cover any fragment or precursors of the polypeptide chain of the protein molecule alone or together with associated molecules or residues linked thereto, e.g., sugar or phosphate residues, or aggregates of the protein molecule or the sugar residues by themselves, provided said active fraction has at least a substantially similar activity to INSP163.

In accordance with the present invention, INSP163 may also be administered to the human body in form of a vector comprising said nucleic acid molecule. Therefore, the invention further relates to the use of a vector comprising said nucleic acid molecule for the manufacture of a medicament for the treatment and/or prevention of lung cancer and/or osteoarthritis. Preferably, the vector is an expression vector, comprising a promoter operably linked to all or part of the coding sequence of INSP163. In a further preferred embodiment, the vector is a gene therapy vector. Gene therapy vectors are known in the art, most of them are virally derived vectors, such as adenoviral or lentiviral vectors.

According to the invention, INSP163 may also be administered to the human body in form of a cell producing and/or secreting INSP163. Therefore, the invention further relates to the use of a cell expressing INSP163 for the manufacture of a medicament for the treatment and/or prevention of lung cancer and/or osteoarthritis or any other cancer and/or musculoskeletal/connective tissue disorder, i.e. to cell therapy for the treatment and/or prevention of lung cancer and/or osteoarthritis or other cancer and/or musculoskeletal/connective tissue disorder. The cell may be a naturally producing INSP163 and/or a transfected cell that produces recombinant INSP163. Preferred are cells expressing and secreting high amounts of the protein, such as over-expressing cells carrying high copy numbers of an expression vector comprising a nucleic acid molecule encoding INSP163.

The invention further relates to a cell comprising a vector comprising a nucleic acid molecule encoding all or part of INSP163 for the preparation of a medicament for treatment and/or prevention of cancer and/or musculoskeletal/connective tissue disorder, in particular of lung cancer and/or osteoarthritis. A cell that has been genetically modified to produce a polypeptide according to the invention is also within the scope of the present invention.

The use of an expression vector for inducing and/or enhancing the endogenous production of INSP163 in a cell normally silent or expressing amounts of the inhibitor which are not sufficient, are also contemplated according to the invention. Thus, the invention makes use of a technology known as endogenous gene activation (EGA) for the production of the desired protein.

According to the invention, INSP163 can be administered alone or in combination with several other therapeutic regimens or agents (e.g. multiple drug regimens) to obtain an additive or synergistic effect for the treatment and/or prevention of cancer or musculoskeletal/connective tissue disorder. Therefore, preferably, the medicament of the invention further comprises:
Interferon, in particular interferon-β, or
A Tumor Necrosis Factor (TNF) antagonist, in particular soluble TNFRs, such as soluble p55 (TBPI) and/or soluble p75 (TBP II), or
An anti-cancer agent, or
An anti-arthritis agent.

The anti-cancer agent is selected from platinum compounds such as cisplatin and carboplatin, vinca alkaloids such as vinorelbine, vincristine, and vinblastine, taxines such as docetaxel and paclitaxel, and various topoisomerase inhibitors.

The anti-arthritis agent is selected from NSAIDs, Acetaminophen, ibuprofen, COX-2 inhibitors (coxibs), corticosteroids such as predisone, Hyaluronic acid, Hyalgan or ARTZ.

All treatments are intended for simultaneous, sequential or separate use.

Pharmaceutical compositions comprising one or more of the above substances, together with INSP163, are within the scope of the present invention.

In yet a further embodiment of the invention, INSP163 is used in combination with a TNF antagonist. TNF antagonists exert their activity in several ways. First, antagonists can bind to or sequester the TNF molecule itself with sufficient affinity and specificity to partially or substantially neutralise the TNF epitope or epitopes responsible for TNF receptor binding (hereinafter termed “sequestering antagonists”). A sequestering antagonist may be, for example, an antibody directed against TNF.

Alternatively, TNF antagonists can inhibit the TNF signalling pathway activated by the cell surface receptor after TNF binding (hereinafter termed “signalling antagonists”). TNF antagonists are easily identified and evaluated by routine screening of candidates for their effect on the activity of native TNF on susceptible cell lines in vitro, for example human B cells, in which TNF causes proliferation and immunoglobulin secretion. The assay contains TNF formulation at varying dilutions of candidate antagonist, e.g. from 0,1 to 100 times the molar amount of TNF used in the assay, and controls with no TNF or only antagonist (Tucci et al., 1992).

Sequestering antagonists are the preferred TNF antagonists to be used according to the present invention. Amongst sequestering antagonists, those polypeptides that bind TNF with high affinity and possess low immunogenicity are preferred. Soluble TNF receptor molecules and neutralising antibodies to TNF are particularly preferred. For example, soluble forms of TNF-RI (p55) and TNF-RII (p75) are useful in the present invention. Truncated forms of these receptors, comprising the extracellular domains of the receptors or functional portions thereof, are more particularly preferred antagonists according to the present invention. Truncated soluble TNF type-I and type-II receptors are described in EP914431, for example.

Truncated forms of the TNF receptors are soluble and have been detected in urine and serum as about 30 kDa or 40 kDa TNF inhibitory binding proteins, which are called TBPI and TBPII, respectively (Engelmann et al., 1990). The simultaneous, sequential, or separate use of INSP163 with the TNF antagonist and/or an Interferon is preferred, according to the invention.

According to the invention, TBPI and TBPII are preferred TNF antagonists to be used in combination with INSP163. Derivatives, fragments, regions and biologically active portions of the receptor molecules functionally resemble the receptor molecules that can also be used in the present invention. Such biologically active equivalent or derivative of the receptor molecule refers to the portion of the polypeptide, or of the sequence encoding the receptor molecule, that is of sufficient size and able to bind TNF with such an affinity that the interaction with the membrane-bound TNF receptor is inhibited or blocked.

In a further preferred embodiment, human soluble TNF-RI (TBPI) is the TNF antagonist to be used according to the invention. The natural and recombinant soluble TNF receptor molecules and methods of their production have been described in the European Patents EP 308 378, EP 398 327 and EP 433 900.

Whilst it may be beneficial to block TNF-α in early stages of the disease, it has been discussed that in later stages, TNF itself may exert a beneficial effect on disease progression (Abraham et al., 2000). Therefore, the invention further relates to a combination of INSP163 and TNF for treatment or prevention of lung cancer and/or osteoarthritis, in particular in advanced stages of disease. TNF-α or TNF-β may be used in accordance with the invention.

The invention further relates to a pharmaceutical composition comprising INSP163, optionally together with one or more pharmaceutically acceptable carriers, diluents or excipients, for the treatment and/or prevention of cancer and/or musculoskeletal/connective tissue disorder, in particular lung cancer and/or osteoarthritis. The pharmaceutical composition may further comprise any of the above-identified further components, and preferably an interferon.

The pharmaceutical composition according to the invention may also comprise a vector comprising a nucleic acid molecule according to the invention, or a cell expressing INSP163.

The active ingredients of the pharmaceutical, i.e. polypeptides, nucleic acids or cells according to the invention, or combinations thereof, as well as the combinations of substances mentioned above, may be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amount of the active protein(s) will be a function of many variables, including the type of receptor, the affinity of the substance according to the invention to its receptor, any residual cytotoxic activity exhibited thereby, the route of administration, the clinical condition of the patient.

A “therapeutically effective amount” is such that when administered, the substance according to the invention results in a beneficial effect on disease development or progression in vivo. The dosage administered, as single or multiple doses, to an individual will vary depending upon a variety of factors, including the pharmacokinetic properties of INSP163, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art.

The dose of the polypeptide according to the invention required will vary from about 0,0001 to 100 mg/kg or about 0.01 to 10 mg/kg or about 0.1 to 5 mg/kg or about 1 to 3 mg/kg, although as noted above this will be subject to a great deal of therapeutic discretion. The medicament of the invention may be administered daily, every other day, or three times per week.

The daily doses are usually given in divided doses or in sustained release form effective to obtain the desired results. Second or subsequent administrations can be performed at a dosage, which is the same, less than or greater than the initial or previous dose administered to the individual. A second or subsequent administration can be administered during or prior to onset of the disease.

The invention further relates to a method for treating and/or preventing cancer and/or musculoskeletal/connective tissue disorder, in particular lung cancer and/or osteoarthritis, comprising administering to a patient in need thereof an effective amount of a substance according to the invention, optionally together with a pharmaceutically acceptable carrier. Alternatively, or additionally, a cell producing INSP163 or a nucleic acid molecule of the invention, optionally comprised in an expression vector, may be administered according to the invention.

The expression vector may be administered systemically. Preferably the expression vector is administered by intramuscular injection. A further preferred route of administration is inhalation, in particular if lung cancer is involved in the disease. Topical administration of an expression vector comprising INSP163 sequences, or of an INSP163 polypeptide according to the invention, is a further preferred route of administration, in particular if there is an involvement of the skin.

The invention further relates to a method for the preparation of a pharmaceutical composition comprising admixing an effective amount of INSP163 with a pharmaceutically acceptable carrier, and to a method of treatment and/or prevention of cancer and/or musculoskeletal/connective tissue disorder comprising administering to a host in need thereof an effective amount of INSP163.

All references cited herein, including journal articles or abstracts, published or unpublished patent applications, issued patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### Cloning and Expression

Cloning of INSP163 and construction of mammalian cell expression vectors for INSP163 are described in Example 4 and Example 5 of International Application No. PCT/GB2004/004544.

### Example 2

### Analysis of INSP63 Gene Expression Levels by Taqman Analysis

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, & dTTP, and DEPC-treated water in a volume of 10 μl. The mixture was incubated at 65° C. for 5 min then chilled on ice for 1 min. The following 10 μl cDNA synthesis mix was prepared in a separate tube: 2 μl 10×RT buffer, 4 μl 25 mM MgCl₂, 2 μl 0.1 M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700).

PCR primers for human INSP163 and the housekeeping control gene glyceraldehyde 3-phosphate dehydrogenase (GAPDH) were designed using the Primer Express software (PE Biosystems). The primers selected were h-INSP163-exon3-4-331F (SEQ ID NO: 49) and h-INSP163-exon3-4-392R (SEQ ID NO: 50). The specificity and the optimal primer concentration to use for the TaqMan analysis were determined by testing the INSP163 gene-specific primers on a series of dilutions of plasmid pCR4-TOPO-INSP163. Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the INSP163 gene in each cDNA sample was expressed as a difference in Ct value, i.e. Delta (δ) Ct=Ct (GAPDH)−Ct (INSP163). Results for each sample were then expressed as a fold difference in the number of cycles required for detectable INSP163 gene expression relative to that for GAPDH, according to the formula Fold Difference=2^{(-δCt)}. Finally, the expression level of the INSP163 gene in each cDNA sample was shown relative to the GAPDH gene expression level, where GAPDH expression level=100%, by dividing 100 by the Fold Difference for INSP163. Results are shown in table 4.

TABLE 4
Expression of INSP163 in various human
tissues as measured by RT-PCR (TaqMan).

| | EXPRESSION |
|---|---|
| | RELATIVE TO |
| CDNA | GAPDH (=100) |
| S76 Brain | 0.05 |
| S77 Heart | 0.07 |
| S78 Kidney | 0.29 |
| S79 liver | 0.00 |
| S80 Lung | 0.08 |
| S81 Placenta | 0.11 |
| S82 skeletal Muscle | 0.00 |
| S83 small intestine | 0.20 |
| S84 Spleen | 0.12 |
| S85 Thymus | 0.03 |
| S86 Uterus | 0.16 |
| S87 Bone Marrow | 0.03 |
| S88 Thyroid | 0.04 |
| S89 Spinal cord | 0.07 |
| S 90 Cervix | 0.27 |
| S91 colon | 0.03 |
| S92 ovary | 0.04 |
| S93 prostate | 0.03 |
| S94 testis | 0.07 |
| S95 skin | 0.50 |
| S113 pancreas | 0.28 |
| S115 Salivary gland | 0.34 |
| S116 Adrenal gland | 0.34 |
| S117 Universal h- ref | 0.03 |
| S119 Breast | 0.00 |
| S120 Stomach | 0.05 |
| S121 Fetal Kidney | 0.54 |
| S122 Eye | 0.08 |
| S123 Mammary gland | 0.04 |
| S124 Ovary | 0.02 |
| S125 Pituitary gland | 0.22 |
| S127 human lupus liver | 0.05 |
| S128 human lupus Lung | 0.16 |
| S129 human lupus Spleen | 0.08 |
| S130 human lupus Kidney | 0.03 |
| S131 cirrhosis liver | 0.03 |
| S132 cirrhosis Lung | 0.00 |
| S133 cirrhosis Spleen | 0.02 |
| S134 cirrhosis Small intestine | 0.13 |
| S135 kidney Tumor | 0.14 |
| S136 Liver Tumor | 0.02 |
| S137 Lung Tumor | 2.56 |
| S142 Fetal Liver | 0.02 |
| S138 colon Tumor | 0.06 |
| S140 Fetal brain | 0.06 |
| S141 Fetal spleen | 0.03 |
| S139 Breast Tumor | 0.01 |
| S143 Fetal Heart | 0.01 |
| S11 mixed RA2 | 0.10 |
| S7 Fibroblast SSCA2 | 0.09 |
| S6 Fibroblast SSc N2 | 0.03 |
| S5 Fibroblast NF2 | 0.21 |
| S4 Fibroblast NF1 | 0.16 |
| S3 Fibroblast Clark N | 0.00 |
| S2 Fibroblast Howard ab | 0.04 |
| S1 Fibroblast AG1518 | 0.02 |
| S151 Disease Brain | 0.46 |
| S150 Throast | 0.02 |
| S149 Blood vessy Artery | 0.03 |
| S148 Appendix | 0.06 |
| S147 Bladder | 0.03 |
| S146 Adipose | 0.02 |
| S145 Lymph Node | 0.03 |
| S144 Fetal Lung | 0.00 |
| S67 mixed small intestine UC 18 | 0.06 |
| S65 mixed small intestine Crohn's 7 | 0.32 |
| S64 mixed small intestine Crohn's 8 | 0.41 |
| S63 mixed small intestine normal int 23 | 0.50 |
| S62 mixed small intestine normal int 21 | 0.37 |
| S52 mixed colon 13073 | 0.03 |
| S50 mixed colon 13224 | 0.21 |
| S29 mixed Lung D | 0.11 |
| S28 mixed Lung C | 0.08 |
| S27 mixed Lung A | 0.06 |
| S19 mixed OA4 | 1.52 |
| S18 Fibroblast LA13 | 0.02 |
| S17 Fibroblast LN14 | 0.02 |
| S16 Fibroblast LAb1 | 0.02 |
| S15 Fibroblast LN1 | 0.05 |
| S13 mixed OA1 | 1.55 |
| S12 mixed RA3 | 0.03 |
| BN5 atherosclerotic plaque Z3 | 0.05 |
| BN3 atherosclerotic plaque Z2 | 0.05 |
| BN1 atherosclerotic plaque Z1 | 0.00 |
| S20 Keratinocytes skin K1 | 0.05 |
| S21 Keratinocytes skin K2 | 0.64 |
| S25 LDC lung | 0.21 |
| S36 THP-1 mono/mac | 0.90 |

Defining a threshold of Expression level of INSP163 relative to GAPDH expression of 1.0, TaqMan expression results show unexpected restricted expression of INSP163 in one lung tumor tissue and in two osteoarthritis tissues. No expression of INSP163 is seen in normal lung tissues (expression level of INSP163 relative to GAPDH is below 0.12). The lung tumor is a bronchogenic carcinoma, more specifically a squamous cell carcinoma.

This specific pattern of expression leads to the conclusion of the involvement of INSP163 in lung cancer and osteoarthritis. These surprising properties characterizing the polynucleotides or the corresponding polypeptides of International Application No. PCT/GB2004/004544 make them particularly suitable for the preparation of a drug or pharmaceutical composition.

### Example 3

### Assay for Determining IKK2 Activity in Non-Small Cell Lung Carcinoma Cells (A549)

Tumor necrosis factor-α(TNFα) is a pleiotropic cytokine with multifunctions including cell activation, differentiation and apoptosis. TNFα exerts both apoptotic and antipoptotic effects in cell-type specific manner. The antiapoptotic effects of TNFα appears to be mediated by the upregulation of NF-κB activity. TNFα induced activation of NF-κB increases the expression of several antiapoptotic proteins that protect cells from cell death. When this pathway is inhibited, TNFα, can potentially induce cell death. Activation of NF-kB is mediated by IKK complex.

An assay has been developed that can measure the activation of IKK2 activity in A549 cells, a human lung carcinoma cell. Since TNFα can induce both pro- and anti-apoptotic pathway, blocking the anti-apoptotic gene expression by cycloheximide can lead to cell death. Once the cells undergo apoptosis, they detach from the culture surface. Upon fixing the cells with crystal violet followed by washing, only live cells are stained and this could be read at 540 nm. Thus, this measure is used to determine cell death in A549 cells.

When A549 cells are treated with TNFα in the presence of cycloheximide, it results in apoptosis. Upon pretreating cells with IL-1β or TNFα, to induce the IKK pathway, thus upregulate the anti-apoptotic genes, it can protect cells from death induced by TNFα+cycloheximide treatment. During the pretreatment step with IL-1β, blocking IKK activity with a specific inhibitor can abolish the protective effect of IL1β on TNFα+cycloheximide mediated cell death. Thus, this property of TNFα signaling is used to monitor IKK activity in A549 cells. The IKK-inhibitor can dose-dependently block IL-1β-mediated protective effect in A549 cells, while with EGF there is no protective effect. The protocol followed for monitoring IKK activity is as follows:
1) A549 Cells are seeded (50,000 cells/well) and cultured overnight,
2) The cells are pretreated with IL-1β (1 ng/ml) or TNF-α with or without the compound, i.e. INSP163 polypeptide, in serum free media for overnight. The compound is a specific inhibitor of IKK activity,
3) The cells are treated with TNF-α and cycloheximide for 8 hours, and
4) Cell death is monitored with crystal violet.

The compound, i.e. INSP163, will induce cell death of non-small cell lung carcinoma cell (A549 cells) in the above-mentioned assay. It is therefore concluded that INSP163 (INSP163 polypeptide) is useful for the treatment and/or prevention of cancer, preferably lung cancer, preferably bronchogenic carcinoma.

### REFERENCES

1. Abraham D J, Shiwen X, Black C M, Sa S, Xu Y, Leask A. J Biol Chem. 2000 May 19; 275(20):15220-5.
2. Altschul S F et al, J Mol Biol, 215, 403-410, 1990
3. Altschul S F et al, Nucleic Acids Res., 25:389-3402, 1997
4. Devereux J et al, Nucleic Acids Res, 12, 387-395, 1984.
5. Engelmann, H., Novick, D., and Wallach, D., 1990, J. Biol. Chem. 265, 1531-1536.
6. Pearson W R, Methods in Enzymology, 183, 63-99, 1990
7. Pearson W R and Lipman D J, Proc Nat Acad Sci USA, 85, 2444-2448, 1988.
8. Tucci, A., James, H., Chicheportiche, R., Bonnefoy, J. Y., Dayer, J. M., and Zubler, R. H., 1992, J. Immunol. 148, 2778-2784.

1-30. (canceled)

31. A method of treating lung cancer comprising administering, to a lung cancer patient, a composition comprising:
a) a pharmaceutically acceptable excipient and a polypeptide; or
b) a pharmaceutically acceptable excipient and a polynucleotide,
wherein said polypeptide is:
i) a polypeptide consisting of SEQ ID NO: 30, or
ii) a polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36, or
iii) a mature form of the polypeptide consisting of SEQ ID NO: 2 or SEQ ID NO: 34, or
iv) a histidine tag form of the polypeptide consisting of SEQ ID NO: 16, SEQ ID NO: 18, SEQ ID NO: 20, SEQ ID NO: 22, SEQ ID NO: 24, SEQ ID NO: 32, SEQ ID NO: 26, SEQ ID NO: 28, SEQ ID NO: 32, or SEQ ID NO: 36, or
v) a cleaved form of the polypeptide consisting of SEQ ID NO: 4, SEQ ID NO: 6, SEQ ID NO: 8, SEQ ID NO: 10, SEQ ID NO: 12, or SEQ ID NO: 14, or
vi) a glycosylated form of the polypeptide, wherein the polypeptide is glycosylated at one or more sites, or
vii) a mutein of any of (a) to (f), wherein the amino acid sequence has at least 40% or 50% or 60% or 70% or 80% or 90% identity to at least one of the sequences in (a) to (f), and retains INSP163 biological activity, or
viii) a mutein of any of (a) to (f) wherein any changes in the amino acid sequence are conservative amino acid substitutions to the amino acid sequences in (a) to (f), and retaining INSP163 biological activity, or
ix) a salt or an isoform, fusion protein, functional derivative, active fraction or circularly permutated derivative of any of (a) to (h); or
said polynucleotide is:
i) a nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35, or
ii) a nucleic acid sequence which hybridizes to the complement of the nucleic acid sequence of (a) under moderately stringent conditions or under highly stringent conditions, or
iii) a nucleic acid sequence of any of (a) or (b) wherein said nucleic acid sequence encodes an amino acid sequence having conservative amino acid substitutions to the amino acid sequences in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

32. The method according to claim 31, wherein said polypeptide is glycosylated at residues 43 and/or 281 of SEQ ID NO: 30.

33. The method according to claim 31, wherein said fusion protein comprises an immunoglobulin (Ig) fusion.

34. The method according to claim 33, wherein the Ig fusion is an Fc fusion.

35. The method according to claim 31, wherein the functional derivative comprises at least one moiety attached to one or more functional groups, which occur as one or more side chains on the amino acid residues.

36. The method according to claim 35, wherein the moiety is a polyethylene moiety.

37. The method according to claim 31, wherein said nucleic acid molecule comprises a vector sequence or is contained in a host cell.

38. The method according to claim 31, wherein said pharmaceutical composition further comprises an interferon, for simultaneous, sequential, or separate use.

39. The method according to claim 38, wherein the interferon is interferon-β.

40. The method according to claim 31, wherein said pharmaceutical composition further comprises a Tumor Necrosis Factor (TNF) antagonist for simultaneous, sequential, or separate use.

41. The method according to claim 40, wherein the TNF antagonist is TBPI and/or TBPII.

42. The method according to claim 31, wherein said pharmaceutical composition further comprises an anti-cancer agent.

43. The method according to claim 42, wherein the anti-cancer agent is selected from the group consisting of platinum compounds, vinca alkaloids, taxines, or topoisomerase inhibitors.

44. A method of treating osteoarthritis comprising administering, to a osteoarthritis patient, a composition comprising:
a) a pharmaceutically acceptable excipient and a polypeptide; or
b) a pharmaceutically acceptable excipient and a polynucleotide,
wherein said polypeptide is:
i) a polypeptide consisting of SEQ ID NO: 30, or
ii) a polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36, or
iii) a mature form of the polypeptide consisting of SEQ ID NO: 2 or SEQ ID NO: 34, or
iv) a histidine tag form of the polypeptide consisting of SEQ ID NO: 16, SEQ ID NO: 18, SEQ ID NO: 20, SEQ ID NO: 22, SEQ ID NO: 24, SEQ ID NO: 32, SEQ ID NO: 26, SEQ ID NO: 28, SEQ ID NO: 32, or SEQ ID NO: 36, or
v) a cleaved form of the polypeptide consisting of SEQ ID NO: 4, SEQ ID NO: 6, SEQ ID NO: 8, SEQ ID NO: 10, SEQ ID NO: 12, or SEQ ID NO: 14, or
vi) a glycosylated form of the polypeptide, wherein the polypeptide is glycosylated at one or more sites, or
vii) a mutein of any of (a) to (f), wherein the amino acid sequence has at least 40% or 50% or 60% or 70% or 80% or 90% identity to at least one of the sequences in (a) to (f), and retains INSP163 biological activity, or
viii) a mutein of any of (a) to (f) wherein any changes in the amino acid sequence are conservative amino acid substitutions to the amino acid sequences in (a) to (f), and retaining INSP163 biological activity, or
ix) a salt or an isoform, fusion protein, functional derivative, active fraction or circularly permutated derivative of any of (a) to (h); or
said polynucleotide is:
i) a nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35, or
ii) a nucleic acid sequence which hybridizes to the complement of the nucleic acid sequence of (a) under moderately stringent conditions or under highly stringent conditions, or
iii) a nucleic acid sequence of any of (a) or (b) wherein said nucleic acid sequence encodes an amino acid sequence having conservative amino acid substitutions to the amino acid sequences in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

45. The method according to claim 44, wherein said polypeptide is glycosylated at residues 43 and/or 281 of SEQ ID NO: 30.

46. The method according to claim 44, wherein said fusion protein comprises an immunoglobulin (Ig) fusion.

47. The method according to claim 46, wherein the Ig fusion is an Fc fusion.

48. The method according to claim 44, wherein the functional derivative comprises at least one moiety attached to one or more functional groups, which occur as one or more side chains on the amino acid residues.

49. The method according to claim 48, wherein the moiety is a polyethylene moiety.

50. The method according to claim 44, wherein said nucleic acid molecule further comprises a vector sequence or is contained in a host cell.

51. The method according to claim 44, wherein said composition further comprises an anti-arthritis agent is selected from the group consisting of NSAIDs, Acetaminophen, ibuprofen, COX-2 inhibitors (coxibs), corticosteroids, Hyaluronic acid, Hyalgan or ARTZ.

52. A method of treating cancer and/or musculoskeletal/connective tissue disorders comprising administering to a patient in need thereof an effective amount of a composition comprising:
a) a pharmaceutically acceptable excipient and a polypeptide; or
b) a pharmaceutically acceptable excipient and a polynucleotide,
wherein said polypeptide is:
i) a polypeptide consisting of SEQ ID NO: 30, or
ii) a polypeptide comprising any of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36, or
iii) a mature form of the polypeptide consisting of SEQ ID NO: 2 or SEQ ID NO: 34, or
iv) a histidine tag form of the polypeptide consisting of SEQ ID NO: 16, SEQ ID NO: 18, SEQ ID NO: 20, SEQ ID NO: 22, SEQ ID NO: 24, SEQ ID NO: 32, SEQ ID NO: 26, SEQ ID NO: 28, SEQ ID NO: 32, or SEQ ID NO: 36, or
v) a cleaved form of the polypeptide consisting of SEQ ID NO: 4, SEQ ID NO: 6, SEQ ID NO: 8, SEQ ID NO: 10, SEQ ID NO: 12, or SEQ ID NO: 14, or
vi) a glycosylated form of the polypeptide, wherein the polypeptide is glycosylated at one or more sites, or
vii) a mutein of any of (a) to (f), wherein the amino acid sequence has at least 40% or 50% or 60% or 70% or 80% or 90% identity to at least one of the sequences in (a) to (f), and retains INSP163 biological activity, or
viii) a mutein of any of (a) to (f) wherein any changes in the amino acid sequence are conservative amino acid substitutions to the amino acid sequences in (a) to (f), and retaining INSP163 biological activity, or
ix) a salt or an isoform, fusion protein, functional derivative, active fraction or circularly permutated derivative of any of (a) to (h); or
said polynucleotide is:
i) a nucleic acid sequence as set forth in any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35, or
ii) a nucleic acid sequence which hybridizes to the complement of the nucleic acid sequence of (a) under moderately stringent conditions or under highly stringent conditions, or
iii) a nucleic acid sequence of any of (a) or (b) wherein said nucleic acid sequence encodes an amino acid sequence having conservative amino acid substitutions to the amino acid sequences in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

53. The method according to claim 52, wherein the cancer is a cancer from blood and lymphatic systems, skin cancer, cancer of digestive systems, cancer of urinary systems, breast cancer, ovarian cancer, gynaecological cancer, choriocarcionoma, brain tumor, bone tumor, carcinoid tumor, nasopharyngeal cancer, retroperitoneal sarcoma, soft tissue tumor, thyroid cancer, cancer of unknown primary site, metastase from primary cancers of the skin, breast, colon, prostate, kidney, thyroid, stomach, cervix, rectum, testis, and bone and from melanoma.

54. The method according to claim 52, wherein the musculoskeletal/connective tissue disorder is a tumor of bones and joints, diffuse connective tissue disease, osteoporosis, arthritis associated with spondylitis, Paget's disease of bone, neurogenic arthropathy, nonarticular rheumatism, avascular necrosis, common foot and ankle disorder, infection of bones and joints, common hand disorder, crystal-induced condition or common sports injury.
